(19) United States
(12) Patent Application Publication
(10) Pub. No.: US 2024/0307293 A1
(43) Pub. Date: Sep. 19, 2024

(54) METHODS AND DEVICES FOR MODULATING ELECTROLYTE EXCRETION

(71) Applicant: AQUAPASS LTD., Or-Akiva (IL)

(72) Inventor: Yaacov Nitzan, Hertzelia (IL)

(21) Appl. No.: 18/575,558

(22) PCT Filed: Jun. 30, 2022
(86) PCT No.: PCT/IB2022/000389

§ 371 Date: Dec. 29, 2023

Related U.S. Application Data
(60) Provisional application No. 63/217,563, filed on Jul. 1, 2021.

(51) Int. Cl. A61K 9/00; A61K 31/4965

(52) U.S. Cl. CPC A61K 9/0014; A61K 31/4965

(57) ABSTRACT

The invention generally relates to methods and systems for controlling electrolyte excretion, e.g., sodium, potassium, chloride and bicarbonate, which is useful to remove body fluid during sweating.

### CROSS-REFERENCE TO RELATED APPLICATIONS

This application claims priority to, and the benefit of, U.S. Provisional Application No. 63/217,563, filed on Jul. 1, 2021, the content of which is incorporated by reference herein in its entirety.

### TECHNICAL FIELD

This invention relates to devices and methods for removing fluid from a body by controlled electrolyte excretion.

### BACKGROUND

Imbalances of fluid within the body are responsible for a variety of pathological conditions, some of which are fatal. Congestive heart failure, for example, occurs when fluid builds up around the heart, preventing the heart from pumping blood effectively. This causes the heart to work harder to eject blood, which further weakens the heart and reduces the ability of the heart to function. The resulting fluid accumulation can lead to additional health conditions, hospitalization, and even death. Accordingly, fluid volume and electrolyte concentration of the body must be tightly controlled.

Mechanisms for regulating body fluid include the renal system and sweating. The kidneys, for example, work to maintain a healthy circulating blood volume and systemic blood pressure, while a major fluid excretory route is sweating. Sweat is nearly isotonic with blood, containing mostly water and sodium chloride. Since sodium is the major determinant of blood osmolality, water and sodium concentration are closely interdependent. In fact, sodium functions as a force that pulls water across fluid compartments (osmotic force). Upon sweating, however, sodium and chloride ions are reabsorbed as sweat flows through the sweat duct, producing a hypo-osmotic sweat on the surface of skin, which limits further fluid removal.

### SUMMARY

This invention relates to methods and devices for managing body fluid of a subject by controlled electrolyte excretion. Specifically, the invention provides methods and devices for efficient removal of fluid (sweat) from skin by enhancing electrolyte excretion of a subject. By enhancing the excretion of electrolytes, such as sodium, certain methods and devices of the invention increase amounts of fluid secreted during sweating. Since an imbalance of body fluid is linked to a variety of pathological conditions, including congestive heart failure, methods and devices for fluid removal described herein can provide non-invasive, therapeutic strategies for managing symptoms of several different chronic illnesses, including edema, chronic heart failure, chronic kidney disease, and hypertension.

Methods and devices of the invention are useful for enhancing electrolyte excretion through the skin. The electrolytes that are excreted can be any one or more of sodium, potassium, chloride, magnesium, calcium, or bicarbonate. Preferred embodiments involve topical administration of one or more chemical agents onto a sweat gland, which advantageously provides direct access to an ion channel for eliciting a therapeutic effect with low toxicity.

The methods and devices described herein are especially suitable for use with fluid transfer devices as described WO/2021/014207, incorporated by reference. For example, the devices and methods described herein are useful with fluid transfer devices that involve forming a micro-environment around a body part (e.g., a leg, torso, arm, etc.) of a subject by dispersing air from a console into a wearable chamber that surrounds the body part. The flow of air with the device is useful to raise an ambient temperature (between 35-50 degrees Celsius) while preferably maintaining a relatively low humidity (e.g., less than 60%), thereby inducing sweat from the subject's body part. The devices and methods described herein are useful to enhance fluid removal through sweat glands by modulating electrolyte excretion.

In some preferred embodiments, methods and devices of the invention involve administering an agent that enhances electrolyte excretion. Rather than administering the agent intravenously or orally, it is an insight of the invention that one or more agents can be administered directly through a subject's sweat gland. Advantageously, this is useful to improve therapeutic efficacy of the agent while minimizing any off target and/or toxic effects since the agent is provided topically at or near the intended target site of the agent. For example, when a sweat gland is stimulated to open by induced sweating, methods and devices of the invention may involve administering one or more chemical agents that target a sweat gland duct locally through the skin. The combination of stimulating sweat production while controlling electrolytes excretion is one basis of the innovation which allows regulation of body fluid volume and electrolyte concentration.

According to one aspect of the invention, provided is a method comprising enhancing a rate of sodium excretion through a surface of skin of a subject by inhibiting a rate of ion reabsorption of a sweat duct. The method involves controlling of the reabsorption rate of the sodium ions by modulating ion channel protein targets in the sweat duct, which is useful to achieve enhancement of sodium secretion in sweat. In preferred embodiments, inhibiting the rate of ion reabsorption includes blocking an ion channel.

For example, an ion channel modulator that targets an epithelial sodium ion channel in a sweat gland can be used to reversibly inhibit the activity of these channels, ideally for a few hours, thus reducing the reabsorption of sodium, and increasing its excretion outside to the skin surface. Inhibiting the channels preferably involves blocking the ion channel. The ion channel can be blocked by administering a diuretic, such as a potassium sparing diuretic to the subject. In preferred embodiments, the diuretic is Amiolride.

Methods of the invention may further involve enhancing a rate of sweat production by the subject. For example, methods include enhancing the rate of sweat production by stimulating a movement of fluid from an interstitial compartment of the subject to the surface of the skin, thereby causing fluid removal from the subject. This may be achieved by requiring that the subject perform an activity, e.g., run, which naturally induces sweating. However, in preferred embodiments the movement of fluid is stimulated with a device (e.g., a chamber) that modulates one or more of airflow, pressure, relative humidity, or temperature within an environment around the surface of the skin.

For example, the device may comprise a chamber that is dimensioned to fit around a body part (e.g., a leg, torso, waist, arm) and modulate an environment around that body part in a manner that induces sweating. The method may further involve supplementing, into the environment around the surface of the skin, one or more negatively charged ions. The one or more negatively charged ions may include bicarbonate or hydroxide.

For example, enhancement of sodium excretion can be enabled by targeting a chloride reabsorption ion channel of a sweat gland, e.g., by targeting the cystic fibrosis transmembrane conductance regulator (CFTR). Failure to absorb NaCl in cystic fibrosis sweat ducts is not only due to the electrochemical effects of chloride ions pulling sodium ions to neutralize the potential difference, but also because the epithelial sodium channel activation depends on a state of CFTR function. Accordingly, inhibition of a CFTR channel by methods and devices of the invention can enhance the excretion of both Na and Cl to the skin surface, thereby facilitating fluid removal and thus treating a symptom of chronic illness.

Methods of the invention are useful for the enhancement of sodium excretion. In some embodiments, methods involve urging positively charged sodium ions towards the skin by adding negatively charged ions to a micro-environment formed around the surface of the skin, e.g., an environment created by a wearable fluid transfer device as described WO/2021/014207, which is incorporated by reference, while sweat is stimulated to further increase an electrochemical potential. In some embodiments, methods of the invention are useful to induce an anion deficit of 10-30 mM between Cl to Na secretion in sweat, thus allowing the addition of negatively charged anions (e.g., HCO3 or OH anions) into the duct to enhance the flow of Na towards the skin rather than its reabsorption in the duct channels.

In some embodiments, methods of the invention involve electrostatically trapping sodium ions with one or more soluble polyanions. The polyanions may be administered such that they diffuse down a sweat duct to a depth of, for example, 0-5 mm, or 0-3 mm, while sweat is stimulated to enhance the sweat sodium concentration. Polyelectrolytes can be used as emulsifiers and clarifying agents in applications in cosmetics, water treatments and other.

Glutamic acid polyanions for example, or negatively charged nano structures, can be used to interact with Na ions to carry those ions in a flow of sweat to the skin surface as neutralized structures. Some embodiments of methods of the invention involve administering a chelating agent to a surface of the skin to attract one or more molecules of sodium within the subject towards the surface of the skin for excretion.

In a different aspect, the invention provides an apparatus for enhancing electrolyte excretion. The apparatus includes a chamber dimensioned to fit around a portion of skin of a subject and a dispenser for topical administration of an agent that enhances electrolyte excretion. For example, the chamber can comprise a chamber of a fluid transfer device, such as one of the fluid transfer devices described in WO/2021/014207, incorporated by reference. The chamber can be dimensioned to fit around a portion of skin of the subject while leaving a volume of air between the skin and a wall of the chamber. Preferably, the chamber includes an inlet and an outlet and is configured such that an air flow that is stimulated by a component of the apparatus can pass through the chamber from the inlet to the outlet.

In preferred embodiments, the apparatus is operable to generate a warm air environment around the skin to thereby elevating the skin temperature to levels of between 33-39 degrees Celsius. The disperser of the apparatus is operable to spray the agent into the chamber allowing the agent to enter a sweat to block sodium reabsorption while sweat is facilitated by environment created by the chamber.

In yet another aspect, the invention provides a device for removing electrolytes from a subject. The device includes a support having a plurality of cathodes dimensioned for insertion into skin of a subject, wherein each one of the plurality of cathodes includes a channel through which an ion can pass. A battery is connected to the plurality of cathodes. During operation, activation of the device causes the excretion of electrolytes from the subject by creating an electrical current that urges positively charged cations through the channels of cathodes.

### BRIEF DESCRIPTION OF THE DRAWINGS

FIG. 1 shows one method of the invention.

FIG. 2 shows a fluid transfer device according to aspects of the invention.

FIG. 3 shows a device for removing electrolytes from a body.

FIG. 4 shows a device for removing electrolytes according to a different embodiment.

FIG. 5 shows a sweat gland.

FIG. 6 shows an enlarged view of a proximal duct of the sweat gland of FIG. 5.

FIG. 7 illustrates the removal of electrolytes with a polyanion.

### DETAILED DESCRIPTION

The invention provides methods and devices for removing fluid from a subject (e.g., a human patient) by controlling electrolyte excretion. The excreted electrolytes can include any one or more of sodium, potassium, chloride, or bicarbonate. Preferred methods of the invention involve administering one or more agents that prevent ion reabsorption. The agents can be administered topically. For example, in preferred embodiments of the invention, one or more agents are topically administered onto a sweat gland, which provides a pathway for the one or more administered agents to an ion channel. Advantageously, this is useful to improve therapeutic efficacy of the one or more agents while minimizing or preventing their toxicity.

For example, one insight of the invention is that a sweat gland provides direct access for topical administration of an agent to an ion channel. Once the sweat gland is stimulated to open, for example, by induced sweating, methods and devices of the invention can target one or more agents to a target ion channel direct through the sweat gland. This is preferred over systemic, oral administration, or cutaneous delivery through the skin. The combination of stimulating sweat production while controlling electrolytes excretion is one basis of the innovation which allows regulation of body fluid volume and electrolyte concentration.

Devices of the invention include fluid transfer devices, such as those described in PCT Application Publication No. WO/2021/014207, incorporated by reference herein in its entirety. The devices can be a home-use or an outpatient clinic device for chronic patients at a risk of developing fluid overload. The device is easily adjustable between treatment areas, is easy to operate and monitor, and is easy to clean and maintain. Embodiments of the device are portable, although the device can be static during treatment episodes. The devices generally include a chamber that can be dimensioned to fit around a portion of skin of the subject while leaving a volume of air between the skin and a wall of the chamber. Preferably, the chamber includes an inlet and an outlet and is configured such that an air flow that is stimulated by a component of the apparatus can pass through the chamber from the inlet to the outlet. The devices allow for controlled removal of fluid via sweat by modulating a micro-environment, e.g., temperature or relative humidity, within the chamber. For example, devices of the invention are operable to generate a warm air environment around the skin to thereby elevating the skin temperature to levels of between 33-39 degrees Celsius. The devices further include a mechanism for dispensing one or more agents onto sweat glands of the subject to modulate sodium reabsorption, and thereby enhance sweating.

Sweating can be regulated during treatment to allow for fluid flow from the skin of about 500 milliliters per day. The invention allows patients to ensure no skin or other heat injuries occur during the treatment and there are no excessive losses of electrolytes or salts that cannot be reabsorbed or digested back. Moreover, sweating is a process that most patients typically have experienced, and adverse effects to the skin are unlikely, even in severe heart failure patients. Fluid transfer from an interstitial and intravascular compartment can be stimulated and enhanced via sweat and electrolyte excretion.

In some embodiments, the invention provides devices for removing electrolyte from a body. The device may include a support with a plurality of electrodes dimensioned of insertion into skin of a subject. The electrodes can deliver a flow of electrons into the body of the subject through the skin to cause the removal of electrolytes, e.g., sodium through channels of the electrodes. In some embodiments, the electrodes may be disposed in a wearable cuff that fits around a body part. In some embodiments, the electrodes may be disposed in a patch that is arranged on a body part. Any suitable electrodes may be used, such as electrodes produced by MicroProbes for Life Science (Gaithersburg, Maryland, USA).

According to one aspect, the invention provides a method comprising enhancing a rate of sodium excretion through a surface of skin of a subject by inhibiting a rate of ion reabsorption of a sweat duct. Inhibiting the rate of ion reabsorption may involve blocking an ion channel. For example, blocking the ion channel may involve administering a diuretic, such as a potassium sparing diuretic to the subject. Preferably, the diuretic is administering topically, e.g., as a spray from an aerosol cannister. The method further involves enhancing a rate of sweat production by the subject. For example, the rate of sweat production may be stimulated up to 100 milliliters/hour, or 200 milliliters/hour, or 300 milliliters/hour, 400 milliliters/hour, 500 milliliters/hour, 600 milliliters/hour, 700 milliliters/hour, 800 milliliters/hour, or more.

Enhancing the rate of sweat production generally involves stimulating a movement of fluid from an interstitial compartment of the subject to the surface of the skin, thereby causing fluid removal from the subject. The movement of fluid can be stimulated with a device that modulates one or more of airflow, pressure, relative humidity, or temperature within an environment around the surface of the skin. The device can be an adjustable chamber. The method may further involve adding, into the chamber, one or more negatively charged ions. For example, the anions introduced by systems of the invention may comprise any one of carbonate, sulphate, bromide, chloride, fluoride, iodide, nitride, oxide, or sulfide ions, or any combination thereof. Preferably, the negative ions involve at least one of bicarbonate or hydroxide.

Methods and systems of the invention include adding negative ions to air that flows through a chamber dimensioned for fitting around a body part to stimulate sweating. Negative ions can be put into the airflow by any number of methods capable of introducing ions into air in advance of the air contacting skin at the site of induced sweating. For example, in some embodiments, negative ions may be introduced into the airflow by an ion-exchange resin or ion-exchange polymer. An ion resin is a resin or polymer that functions as a medium for ion exchange and is generally in the form of porous microbeads. The ion resin may be either strongly or weakly basic. In certain instances, methods and systems of the invention include placing the ion resin at an inlet of the chamber such that as air flows through the inlet, the air passes through the ion resin to incorporate negative ions into the airflow. Ion resins can be purchased commercially or may be synthesized, for example, as described in Japanese Patent Application JP2002302665A, incorporated by reference.

Other methods for attracting the sodium molecules to the skin surface can be the use of a chelating agent. Chelating agents are usually used to remove toxic metals from the body and have a ring-like center which forms at least two bonds with the metal ion allowing it to be excreted. For example, cations such as Na may react as competing ions for example on Fe- and Cu-chelates such as EDTA, DTPA and EDDHA. As Na is the dominant cation in the sweat duct, a chelator with affinity for sodium ions can be used to trap the sodium ions and enhance the excreted sweat sodium concentration.

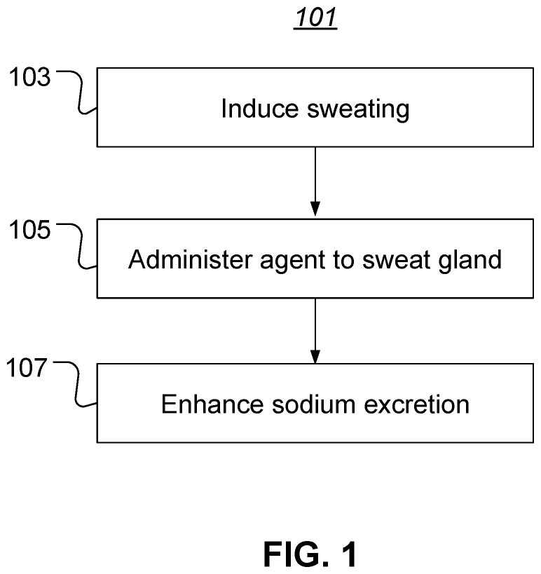

FIG. 1 shows one method 101 of the invention. The method 101 involves inducing 103 sweating from a subject. Sweating is preferably induced 103 with a fluid transfer device, such as the fluid transfer device described below. For example, sweating can be induced 103 by providing an airflow that raises an ambient temperature (between 35-50 degrees Celsius) inside a wearable chamber while keeping the relative humidity low (e.g., less than 60%) to stimulate the sweat glands. The relative humidity reduced by increasing the rate of air flow. By stimulating the production of sweat, the sweat glands are open, filled with interstitial fluid following a few hours of a procedure, and can be used to administer 105 an agent to a target gland duct locally rather than by systemic oral administration or cutaneous delivery through the skin. The combination of stimulating sweat production while controlling electrolytes excretion is the basis for the innovation which allows to regulate the extracellular sodium concentration.

One preferred agent is the epithelial sodium channel (ENaC) blocker Amiolride. Amiloride, which may be found under the trade name Midamor, is a medication typically used with other medications to treat high blood pressure or swelling due to heart failure or cirrhosis of the liver. Amiloride is classified as a potassium sparing diuretic. Amiloride is often used together with another diuretic, such as a thiazide or loop diuretic. It is taken by mouth, however, it is preferred that the agent is administered topically to a sweat gland for faster, more effective therapeutic effect. Onset of action is about 15 minutes to able one hour or two hours.

Amiloride can be applied directly on the skin through lotion or gel. Also, it can be complexed in delivery vehicles (e.g., liposomes or micro-vesicles) to improve its penetration and cargo stability through the sweat duct. Even if Amiloride administered orally, its effect on sodium excretion is very different then used today, given as a diuretic medication to influence the kidney reabsorption. In this invention setting, its systemic oral administration can be in conjunction with sweat gland activation, allowing enhanced excretion of sodium also through the skin.

Some embodiments of the invention involve targeting a cystic fibrosis transmembrane conductance regulator (CFTR) protein, often referred to as an ion channel. The CFTR protein helps to maintain a healthy balance of salt and water on many surfaces in the body, including the skin. When the protein is not working correctly, chloride—a component of salt—becomes trapped in cells. Without the proper movement of chloride, water cannot hydrate the cellular surface.

In particular, the CFTR protein is a particular type of protein called an ion channel. An ion channel moves atoms or molecules that have an electrical charge from inside the cell to outside, or from outside the cell to inside. Accordingly, the same channel, i.e., the CFTR channel, is common to fluid transport functions that are oppositely directed, i.e., secretion and absorption. In the sweat gland, salt absorption via the CFTR channel can occur. Cl ions can enter the cell from a lumen. The CFTR ion channel can move chloride ions from inside the cell to outside the cell. To get out of the cell, the chloride ions can move through a center of a tube formed by CFTR proteins. Once the chloride ions are outside the cell, osmotic forces increase, thereby attracting and moving molecules of water. Accordingly, some embodiment of the invention involves targeting CFTR proteins to enhance excretion of chloride ions from the body, which facilities the removal of water through the subject's skin.

In some embodiments, the method 101 involves delivering spironolactone via a topical agent or via an aerosol onto an eccrine sweat gland. Spironolactone can travel into the body via the sweat gland to inhibit sodium reabsorption in the eccrine sweat gland duct. Methods may also involve administering a rinse or spray the skin comprising an acid (e.g., hypochlorous acid), which can enter the duct and remove more sodium once sweated out.

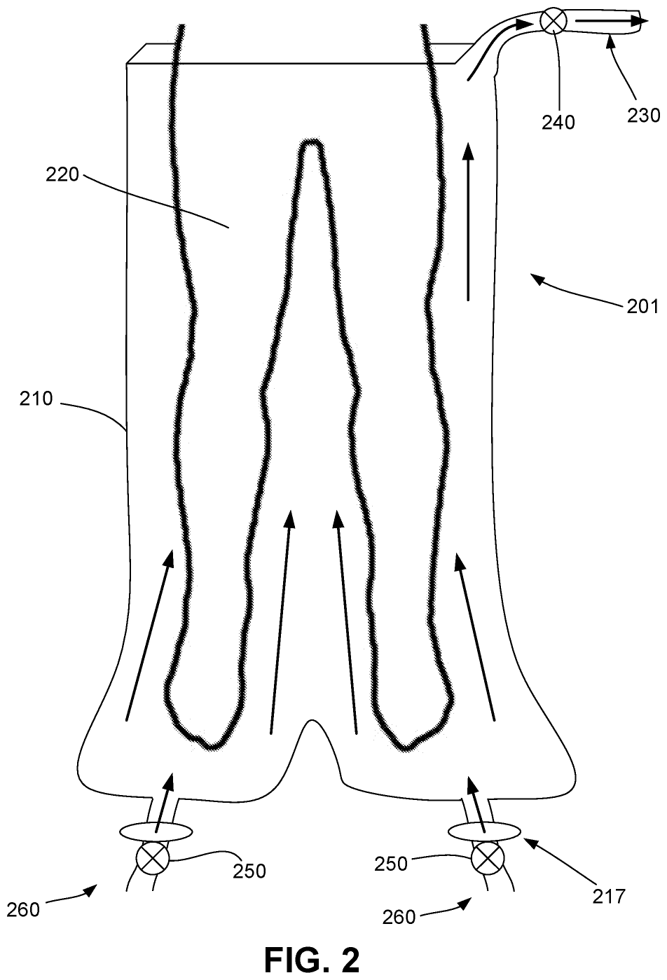

FIG. 2 shows a fluid transfer device 201 according to aspects of the invention. The device 201 includes a warm air cuff 210 dimensioned for fitting around the legs 220 of a subject. The cuff 210 has one or more hot air inlets 260 and one or more outlets 230. A relative humidity sensor 250 can be disposed at the hot air inlet 260. Dry warm air at a temperature of about 32° C. to about 45° C. at a relative low humidity of less than about 85% is fanned into the warm air cuff 210 at the hot air inlet 260. The relative low humidity may, for example, be about 85%, 80%, 75%, 70%, 65%, 60%, 55%, 50%, 45%, 40%, 35%, 30%, 25%, 20%, 15%, 10%, or 5%. Air and sweat are exhausted from the cuff 210 at the outlet 230. A second relative humidity sensor 240 can disposed at the outlet 230. An agent dispenser 217 can be disposed at the inlet. The dispenser can be an aerosol cannister having an agent, e.g., a diuretic, for stimulating the excretion of an electrolyte. For example, the device can stimulate the induction of sweat and then deliver an agent that causes the excretion of an electrolyte. The electrolyte may be any one or more of calcium, chloride, magnesium, phosphate, potassium, sodium. Preferably sodium.

In some embodiments, the chamber is sized to fit around a patient's abdomen, one or two legs, one or two arms, a back, or any combination thereof. In some instances, the chamber may cover a substantial portion of the patient's body below the chest, for example, covering around 1 square meter of surface area and providing fluid loss at a rate of approximately 200 ml/hr.

The patient can request, or be prescribed, a certain amount of sweat per treatment. The systems of the invention will calculate sweat rate, display, and stop the operation of the system once the desired sweat amount is obtained. Calculation can be performed using input from the two humidity sensors and their difference in readings over time, such as every few minutes. In another embodiment, the invention is directed to removing fluids from the body in order to maintain intravascular fluid balance by driving fluids through the skin by osmosis, either alone or in combination with sweat production. A cuff with high concentration of intravascular large molecules can be tight around the body legs arms or abdominal cavities and through a semipermeable cuff water or dry air will start to flow out through the skin and into the cuff. This way, fluids can shift out of the body and fluid balance can be maintained. This method can be done by the patient at home and eliminate episodes of severe fluid overload.

Sweat rates, when the skin is exposed to local, tolerable temperature elevations of about 1° C. to about 5° C., can be in the rate of about 0.4 milligram per cm2 per minute. Such a rate translates to a sweat rate of over about 200 milliliters/hour from one limb having a surface area of around 10,000 cm2 which is the average body surface area of both legs and torso. The sweat rate is calculated using the following formula: sweat rate=absolute humidity out-absolute humidity in ×airflow

The invention may be used for patients of different ages. As reported in a study from Miranda A. Farage et al., Textbook of Aging Skin, thermoregulation effects were evaluated for a group of younger subjects (21-39 years in age) and a group of older subjects (61-73 years in age). Sweat responses, esophageal, skin temperatures, non-evaporative heat exchange, heart rate, cardiac output, blood pressure, forearm blood flow, and metabolic heat production were examined after exposing the subjects to 40° C. and 40% relative humidity for up to 130 minutes. The study reported that there was no significant difference in sweat rate or onset of sweating between the groups. Therefore, with age, sweat rates and changes with sweat rates can still be high enough to promote decongestion treatment in patients chronically overloaded with fluid. Sweat rate is typically not affected by medical conditions such as congestive heart failure (CHF).

Furthermore, sweat rate is not reduced in CHF patients. Sweat rate may even be higher in CIF patients because SSNA activity is not altered compared to a control. Whole body heating induces significant sweating responses, while the sweat rate toward the end of a moderate whole-body heating in CHF patients is marginally lower (˜20%) than that of control subjects. In CHF patients, SSNA and sweat rate increased during the initial period of whole body heating (e.g., internal temperature increase ˜0.2-0.3° C.), while neither increased in healthy controls during this period.

In the invention, local heat provides an environment that allows for sweating of the body part where sweat evaporates quickly from the skin and the environment keeps the skin moist and well-nourished during the procedure. Sweat can be stimulated to result in a local limb fluid removal rate of over about 150 milliliters/hour. Treatments for the local body part can be short in duration and administered throughout the day, resulting in treatment of about a few hours overall in a day.

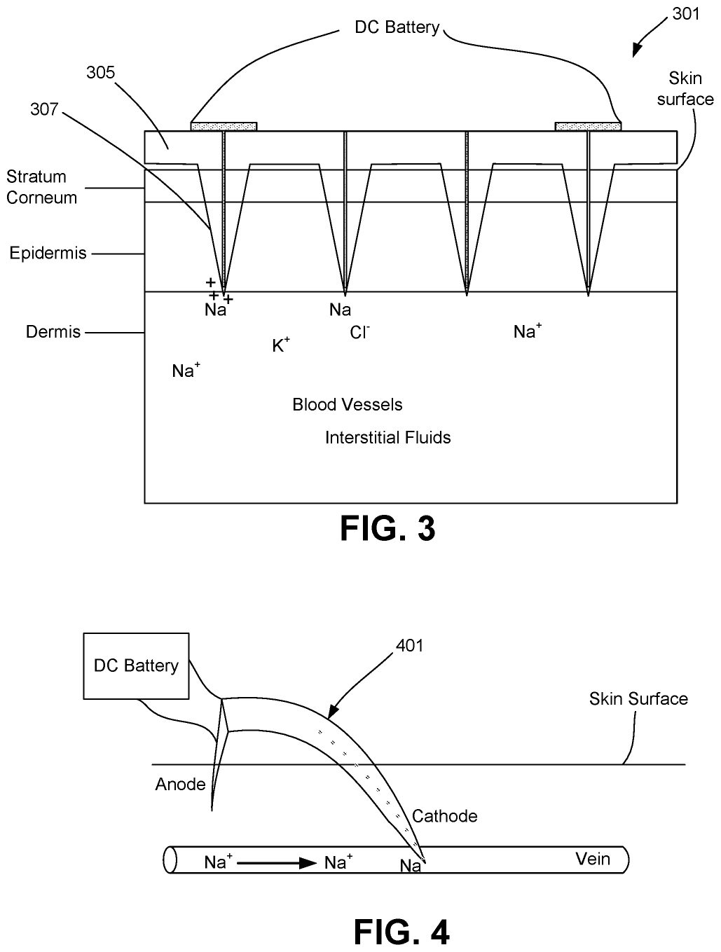

FIG. 3 shows a device 301 for removing electrolytes from a body. The device 301 includes a support 305 having a plurality of cathodes 307 dimensioned of insertion into skin of a subject. The plurality of cathodes 307 protrude outward from a surface of the support for insertion through a surface of skin. The device 301 includes a battery for activating the device and providing a flow of electrons through the cathodes and into the body of the subject through the skin to for the removal of electrolytes, e.g., sodium, through channels of the cathodes.

During operation, the plurality of cathodes 307 can provide a negatively charged surface within the interstitial fluid or blood vessels of a subject. For example, the plurality of cathodes 307 can be introduced to a location having interstitial fluids containing electrolytes. The negatively charged surface of the plurality of cathodes 307 is useful to attract positively charged electrolytes (e.g., sodium) from the interstitial fluids or blood vessels for extraction. In particular, each of the plurality of cathodes includes a channel dimensioned for receiving positively charged electrolytes. When a current is activation, the cathodes facilitate the removal of positively charged electrolytes through the channels, which enhances fluid removal by sweat. In some embodiments, device 301 is a component of a wearable cuff that fits around a body part. In some embodiments, the cathodes may be disposed in a patch that is arranged on a body part.

In some embodiments of sodium removal, reverse iontophoresis is performed by applying 1-2 patches, or more, of electrodes onto skin without penetrating needles. At least one patch being an anode and at least one other patch being a cathode. The area of the patch can be between 1-500 square cm, for example, about 25, 50, 75, 100, 150, 200, 250, 300, 350, 400, 450, or 500 square cm. Preferably the patch comprises a rectangular shape and is configured to be placed on any body part, e.g., a leg, an arm, a torso. In preferred embodiments, multiple patches are used, for example, multiple pairs of patches having both an anode and cathode. The pairs of patches can be placed on one body part, or multiple body parts. Preferably the patches are placed on multiple body parts, for example, substantially of all of a subject's body parts, including the subject's arms, legs, and torso. The reverse iontophoresis can be performed by applying a small current in the region of 0.1-10 milliamperes, e.g., about 1, 2, 3, 4, 5, 6, 7, 8, 9, or 10 milliamperes. The Na+ will be attracted to the cathode at a rate of approximately 5 micromoles per hour per square cm of cathode. This will yield Na extraction of around 20-200 mmol/hr which is clinically significant and will be very efficient in decongestion of heart failure patients patients especially when treated with any type of fluid removal system in which the removed fluids are hypotonic such as with a fluid transfer device, such as the fluid transfer device provided under the tradename Aquapass, and as described in WO/2021/014207, which is incorporated by reference.

When used with the fluid transfer device the wetted skin from the enhanced sweat will increase the reverse iontophoresis conductivity and drive the Na+ faster out from the skin. The Na+ from the other body parts will be driven to the extraction area either through direct interstitial fluid flow or through the intravascular blood flow.

FIG. 4 shows a different device 401 for removing electrolytes. The device 401 includes a cathode dimensioned for insertion into a blood vein of a subject. The cathode includes a pointed distal end for insertion through skin of a subject, and a channel for actively transporting electrolytes (e.g., sodium) from the vein and outside the body. The removal of positively charged electrolytes is useful to facilitate water removal during sweating.

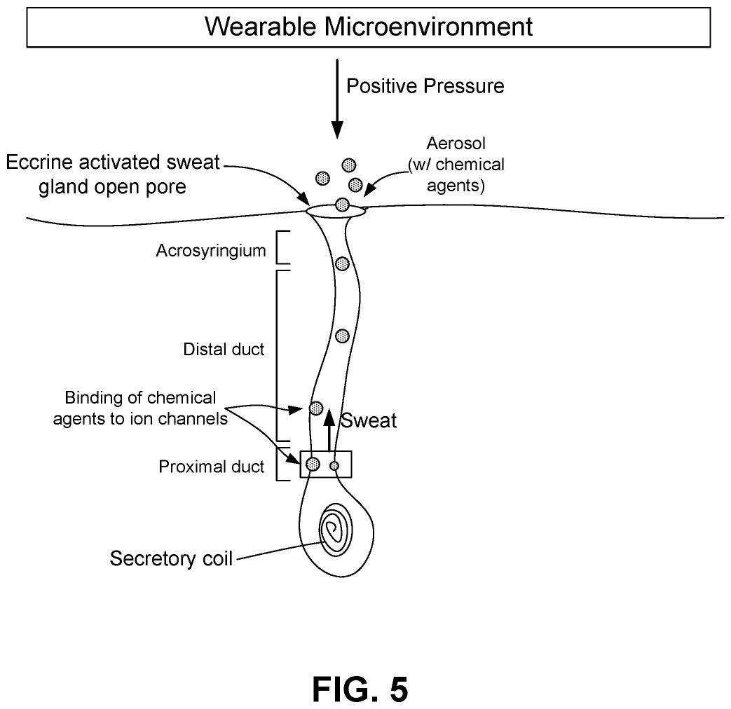

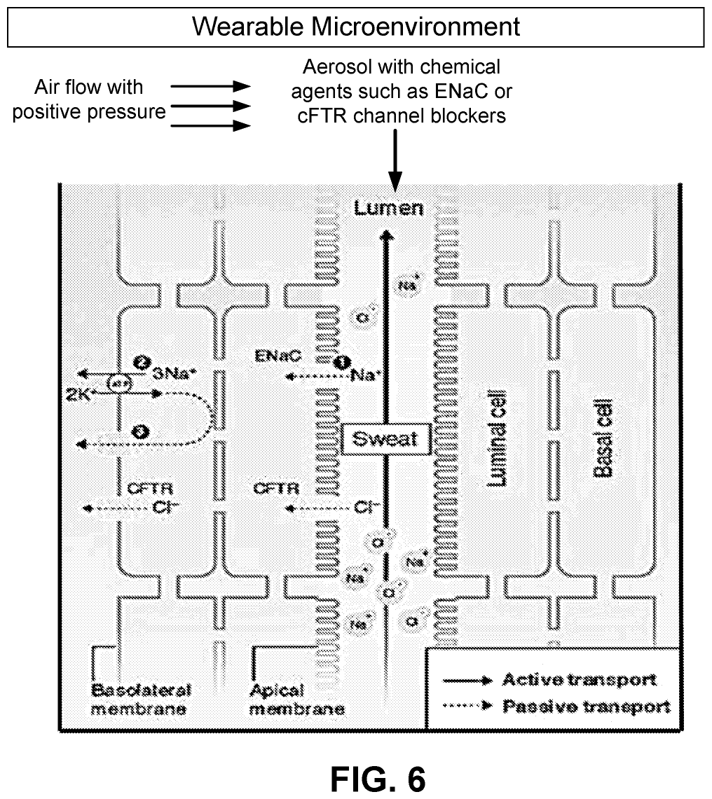

FIG. 5 shows a sweat gland and FIG. 6 shows an enlarged view of the proximal duct of the sweat gland of FIG. 5. The sweat gland comprises a distal duct, and a proximal duct. An agent topically applied to the sweat duct can travel down the distal duct to the proximal duct. At or near the proximal duct, the agent may bind with an ion channel. Binding of the agent to the ion channel is useful to effect excretion of an electrolyte, such as sodium.

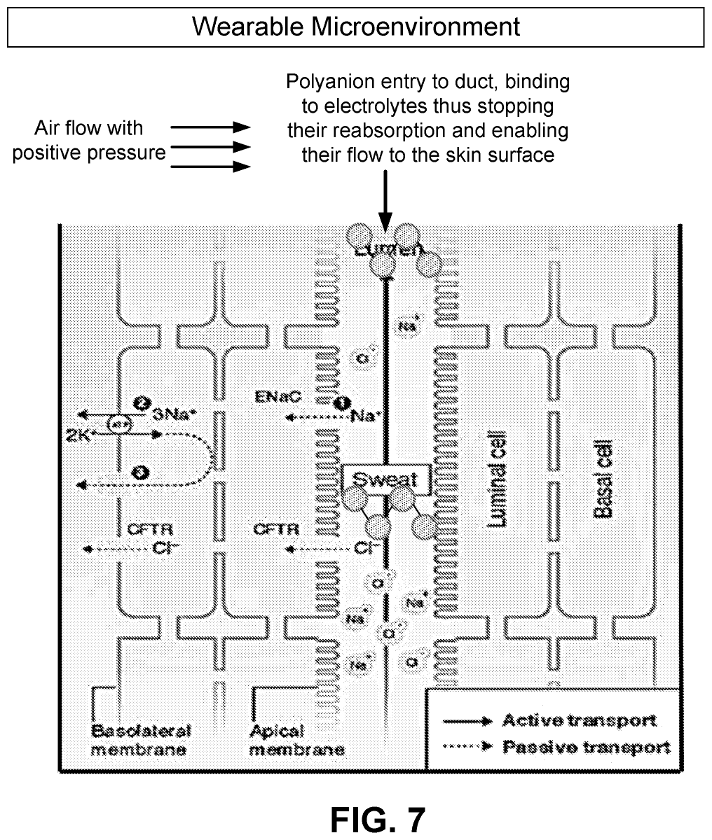

FIG. 7 illustrates the removal of electrolytes with a polyanion. Sodium ions, for example, can be electrostatically trapped by soluble polyanions that are administered to a surface of skin and diffuse down a sweat duct, for example, to a depth of 0.001-3 millimeters, while sweat is stimulated to enhance the sweat sodium concentration of sweat, i.e., prevent reabsorption. Polyelectrolytes are traditionally used as emulsifiers and clarifying agents in applications in cosmetics, water treatments and other. Glutamic acid polyanions, for example, or negatively charged nano structures, can be used in a similar manner to interact with the sodium ions, but instead of forming precipitate to stop the sweat flow, the polyanions of the invention are useful to transport sodium ions upstream with a sweat flow to the skin surface as neutralized structures.

As previously described, an example for such an apparatus can be the addition of the negatively charged ion solution directly on the skin surface before or during sweat stimulation, or alternatively with positive pressure generated by the wearable.

Methods and devices of the invention are useful to treat fluid imbalances related to body osmolality. For example, an increase in body osmolality can trigger compensatory mechanisms that cause water retention and edema. The excess fluid, primarily salt and water, builds up and leads to an increase in weight and swelling. Methods and devices of the invention can treat edema by removing water through skin while inhibiting sodium reabsorption, which enchances the removal of water through the skin.

Currently, ways to control electrolytes concentration involve diet restriction, systemic medications that target the kidneys to either dispose of or preserve electrolyte concentration or mechanical invasive solute exchange such as in dialysis. Nevertheless, it is difficult to effectively control serum sodium concentration and reduce interstitial fluid accumulation in order to control blood volume expansion in patients. There are no commercially available ways to control electrolyte concentrations through sweat. Thus, a method and apparatus to control electrolytes excretion to the skin surface directly from the interstitial compartment can be greatly beneficial to treat pathologies for example such as heart Failure (HF), chronic kidney disease (CKD) and hypertension.

Body fluid volume and electrolyte concentration are normally maintained within very narrow limits despite wide variations in dietary intake, metabolic activity, and environmental stresses. Homeostasis of body fluids is preserved primarily by the kidneys in order to maintain an effective circulating blood volume and systemic blood pressure. Sodium is the major determinant of plasma osmolality, thus water and sodium balance are closely interdependent. Sodium acts as a force that pulls water across the fluid compartments (the osmotic force). A steady-state is achieved when the plasmatic osmolality is 1 mOsm/L greater than the interstitial space, and the capillary hydrostatic pressure opposes the osmotic movement of the water into the intravascular space.

As described, another excretory route, similar to that of the renal system, is perceived through sweating supplied directly from the interstitial compartment. Sweat glands, beside their thermoregulatory role, have other homeostatic functions such as clearing excess micronutrients, metabolic waste and toxicants. Eccrine glands are the major sweat glands located throughout the body with open access to the atmosphere through the sweat pores.

Primary sweat is nearly isotonic with blood plasma with sodium (Na) concentrations of around 140 mmol/liter, however Na and Cl ions are reabsorbed as sweat flows through the sweat duct, producing hypo-osmotic skin surface sweat. Chloride (Cl) secretion in sweat generally follows that of Na except that its concentration is lower by 10-30 mM. The remaining anion deficit is calculated to be 15-20 mM lactic anion and approximately 10-15 mM bicarbonate (HCO3) concentration in the precursor sweat, a value half that of plasma HCO3. HCO3 concentration in the skin surface sweat of the adult human ranges from 2 to 10 mM and tends to increase with the sweat rate.

Most of sweat is water and NaCl, but also low concentration of proteins as well as low levels of solutes such as potassium, calcium, lactate, urea, bicarbonate, amino-acids and peptides originate from the interstitial fluid. In addition to the above, sweat also contains magnesium, iodide, phosphorus, sulfate, and metals such as iron, zinc, copper, cobalt, lead, manganese, molybdenum, tin, and mercury in trace amounts.

Humans have the capability of producing a large amount of sweat under certain physiological conditions, up to 2 L/hr of filtrated fluids. According to THAYSEN'S hypothesis, there is a limited capacity for Na reabsorption by the sweat duct, and after the re-absorptive capacity is saturated (e.g., at high sweat rate), the further increase in Na is proportional to the increase in flow rate. Thus, depending on the sweat rate, sodium secretion rate can be ranged between 10 mmol/Liter-100 mmol/Liter.

The mechanism of sweat gland secretion and electrolytes reabsorption is well established. The sweat duct is composed of homogeneously similar cells dedicated to salt absorption, with CFTR (cAMP-regulated Cl channel) and ENaC (Na ion channel) as the only active conductance in the apical membrane. Loss-of-function mutations in CFTR cause the genetic disease cystic fibrosis resulting in excessive salty sweat.

Most of the NaCl reabsorption occurs in the proximal duct by the apical and basolateral membranes as these cells contain more mitochondria and Na—K-ATPase activity than that of the distal segment of the eccrine duct.

The invention generally relates to methods and systems for controlling electrolytes such as sodium, potassium, chloride and bicarbonate concentration by enhancing their excretion to the skin surface through sweating. It describes methods and apparatus for topical administration of chemical agents through activated eccrine sweat glands, enabling their entrance by diffusion or positive pressure gradient down the sweat gland counter to the upstream hydrostatic pressure.

The method includes control of the reabsorption rate of the sodium ions by modulating ion channel protein targets in the sweat duct to achieve enhancement of sodium secretion in sweat (as in Cystic Fibrosis and other genetic diseases in which sodium reabsorption is reduced by non-functional ion channels).

For example, an ion channel modulator that potentially target the ENaC ion channels in the sweat gland will reversibly inhibit the activity of these channels, ideally for a few hours, thus reducing the reabsorption of sodium, and increase its excretion outside to the skin surface. Enhancement of sodium excretion is also enabled through targeting the chloride reabsorption ion channel in the sweat gland, the CFTR. Failure to absorb NaCl in cystic Fibrosis sweat ducts is not only due to the electrochemical effects of chloride ions pulling sodium ions to neutralize the potential difference, but also because the ENaC activation depends critically on the state of CFTR function. Inhibition of the CFTR channel will enhance the excretion of both Na and Cl to the skin surface.

An example for such an apparatus could be the use of a pharmacological agent topically administered to the skin before or during procedure. One example for such an agent is the ENaC blocker Amiolride, a sodium absorption inhibitor which is a potassium sparing diuretic typically used to treat high blood pressure or swelling due to heart failure or cirrhosis of the liver. Amiloride can be applied directly on the skin through lotion or gel. Also, it can be complexed in delivery vehicles to improve its penetration and cargo stability through the sweat duct. Even if Amiloride administered orally, its effect on sodium excretion is very different then used today, given as a diuretic medication to influence the kidney reabsorption. In this invention setting, its systemic oral administration will be in conjunction with sweat gland activation, allowing enhanced excretion of sodium also through the skin.

Another apparatus could be topical administration of a pharmacological agent such as an Amiloride via an aerosol that is sprayed by the air inflow line of the air generator that is used to increase the skin temperature to initiate the sweating. Positive pressure within the wearable's microenvironment can be further generated at the onset of sweat stimulation or alternatively when the sweat stops flowing as a result of temporary reduction in temperature. At this timing, the duct is open, as there is fluid within it, however its pressure is down to zero as the fluid is reabsorbed back into the body. The device can generate pressures up to 20 mmHg above atmospheric pressure that can help drive the pharmacological agent into the duct. This specific timing can be recognized by the sweat rate indicated online by the device.

Other method includes inhibition of the hormone Aldosterone in the eccrine sweat gland. Plasma Aldosterone upregulates sweat sodium excretion by influencing the Na—K-ATPase activity which controls the reabsorption of Na+. The concentration of sodium and chloride in sweat is greatly elevated in conditions of chronic deficiency of adrenal secretions, for example, Addison's disease in man. Aldosterone blockage with spironolactone, a potassium-sparing diuretic, can be used to increase sweat sodium concentration.

Other methods of the invention involve targeting the activation of the protease Corin to promote sweat and salt excretion. Corin is a protease that converts pro-ANP to ANP with the main function of causing a reduction in expanded extracellular fluid (ECF) volume by inhibiting ENaC sodium channels in the kidney and in the sweat glands. Activation of the natriuretic peptide receptor-A (NPR-A) by delivery, for example, of ANP locally to the sweat gland pore through the AquaPass system, i.e., a fluid transfer device as described in WO WO/2021/014207, incorporated by reference, can be used to increase sodium excretion through a similar corin-ANP-mediated ENaC inhibitory mechanism.

Some embodiments of the invention involve targeting a cystic fibrosis transmembrane conductance regulator (CFTR) protein ion channel. This ion channel is acutely regulated through interaction of kinase and phosphatase for sustained Cl absorption in the face of rapidly changing electrochemical gradients for Cl. Application of the protein kinase inhibitor staurosporine or removal of either substrate ATP or Mg 2+ cofactor can be used to block cAMP activation of the CFTR channel.

Other method for enhancement of sodium excretion is to pull the positively charged sodium ions towards the skin by adding negatively charged ions to the micro-environment of the wearable while sweat is stimulated to further increase the electrochemical potential. Anion deficit of 10-30 mM exists between Cl to Na secretion in sweat, thus allowing the addition of HCO3 or OH anions for example into the duct to enhance the flow of Na towards the skin rather than its reabsorption in the duct channels.

As previously described, an example for such an apparatus can be the addition of the negatively charged ion solution directly on the skin surface before or during sweat stimulation, or alternatively with positive pressure generated by the wearable.

Other methods for attracting the sodium molecules to the skin surface can be the use of a chelating agent. Chelating agents are usually used to remove toxic metals from the body and have a ring-like center which forms at least two bonds with the metal ion allowing it to be excreted. It is thought that cations such as Na may react as competing ions for example on Fe- and Cu-chelates such as EDTA, DTPA and EDDHA. As Na+ is the dominant cation in the sweat duct, a chelator with affinity for sodium ions can be used to trap the sodium ions and enhance the excreted sweat sodium concentration.

In some aspects, the invention provides a method to reduce sodium concentrations in the interstitial compartment and regulate sodium concentration in the plasma by enhancing sweat rate and sodium excretion to the skin surface through modulating ion channel protein targets in the sweat duct to reversely inhibit the reabsorption rate of the sodium ions. An apparatus for topical administration of a lotion or gel with a pharmacological agent or a soluble polyanion directly on the skin.

In some aspects, the invention provides an apparatus for enhancing sweat using warm air generated into a wearable around the body elevating the skin temperature to levels of between 33-39 degrees Celsius. In such an apparatus, within a warm humid air inline pipe, there is a disposable apparatus that includes a disperser with a soluble pharmacological agent sprayed into the humid air as an aerosol. As the air flows through the disperser, the air becomes full of the agent aerosol solution and enters through the sweat gland pores deep enough into the duct to reach its ion channel protein targets and block sodium reabsorption.

In some aspects, the invention provides a method to trap sodium ions and to increase their electrochemical pull by adding a soluble polyanion that can diffuse down the sweat duct whilst sweat is stimulated counterflow to electrolytes to increase the sweat sodium concentration.

### INCORPORATION BY REFERENCE

References and citations to other documents, such as patents, patent applications, patent publications, journals, books, papers, web contents, that have been made throughout this disclosure are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

What is claimed is:

1. A method comprising enhancing a rate of sodium excretion through a surface of skin of a subject by inhibiting a rate of ion reabsorption of a sweat duct.

2. The method of claim 1, wherein inhibiting the rate of ion reabsorption comprises blocking an ion channel.

3. The method of claim 2, wherein blocking the ion channel involves administering a potassium sparing diuretic to the subject.

4. The method of claim 1, further comprising enhancing a rate of sweat production by the subject.

5. The method of claim 4, wherein enhancing the rate of sweat production comprises stimulating a movement of fluid from an interstitial compartment of the subject to the surface of the skin, thereby causing fluid removal from the subject.

6. The method of claim 5, wherein the movement of fluid is stimulated with a device that modulates one or more of airflow, pressure, relative humidity, or temperature within an environment around the surface of the skin.

7. The method of claim 6, wherein the device comprises a chamber.

8. The method of claim 6, wherein the method further comprises supplementing the environment around the surface of the skin with one or more negatively charged ions.

9. The method of claim 8, wherein the one or more negatively charged ions comprise bicarbonate or hydroxide.

10. The method of claim 1, further comprising administering a chelating agent to the surface of the skin to attract one or more molecules of sodium within the subject towards the surface of the skin for excretion.

11. The method of claim 1, further comprising administering a polyanion down a channel of a sweat duct.

12. The method of claim 1, wherein the ion is a sodium ion.

13. The method of claim 1, wherein the ion is a chloride ion.

14. An apparatus comprising:
a chamber dimensioned to fit around a portion of skin of a subject; and
a dispenser for topical administration of an agent that enhances electrolyte excretion.

15. The apparatus of claim 14, wherein the chamber is dimensioned to fit around the portion of skin of the subject while leaving clear a volume of air between the skin and a wall of the chamber.

16. The apparatus of claim 14, wherein the chamber comprises an inlet and an outlet and the chamber is configured such that an air flow that is stimulated by a component of the apparatus passes through the chamber from the inlet to the outlet.

17. The apparatus of claim 14, wherein the apparatus generates a warm air environment around the skin thereby elevating the skin temperature to levels of between 33-39 degrees Celsius.

18. The apparatus of claim 14, wherein the disperser is operable to spray the agent into the chamber allowing the agent to enter a sweat to block sodium reabsorption while sweat is facilitated by environment created by the chamber.

19. A device comprising:
a support comprising a plurality of cathodes dimensioned for insertion into skin of a subject, each of the plurality of cathodes comprising a channel through which an ion can pass; and
a battery connected to the plurality of cathodes.

20. The device of claim 19, wherein, during operation, activation of the device causes the excretion of ions from the subject by creating an electrical current that urges positively charged ions through the channels of cathodes.
